(19) United States
(12) Patent Application Publication
(10) Pub. No.: US 2020/0397945 A1
(43) Pub. Date: Dec. 24, 2020

(54) Biological Formulations and Methods for Treating Cardiac Tissue and Disorders

(71) Applicant: CorMatrix Cardiovascular, Inc., Roswell, GA (US)

(72) Inventor: Robert G Matheny, Norcross, GA (US)

(21) Appl. No.: 16/990,576

(22) Filed: Aug. 11, 2020

Related U.S. Application Data
(63) Continuation-in-part of application No. 16/531,263, filed on Aug. 5, 2019.
Application No. 16/531,263 is a continuation of application No. 15/877,586, filed on Jan. 23, 2018, now Pat. No. 10,383,977.
Application No. 15/877,586 is a division of application No. 15/386,640, filed on Dec. 21, 2016, now Pat. No. 10,143,778.
Application No. 15/386,640 is a continuation-in-part of application No. 13/328,287, filed on Dec. 16, 2011, now Pat. No. 9,532,943.

(51) Int. Cl. A61L 27/36; A61F 2/06; A61F 2/00; A61L 27/34; A61L 27/54; A61L 27/58; A61K 31/4418; A61L 27/38; A61L 27/50

(52) U.S. Cl. CPC A61L 27/3633; A61F 2/06; A61F 2/0095; A61L 27/34; A61L 27/54; A61L 27/58; A61L 2300/414; A61L 27/3834; A61L 27/507; A61F 2250/0067; A61F 2210/0004; A61L 2430/20; A61K 31/4418

(57) ABSTRACT

Methods for treating damaged cardiac tissue by delivering biological formulations proximate the pericardial space of a mammalian heart that (i) enhance and supplement the properties provided by the GATA6⁺ macrophages in the serous fluid and/or (ii) restore, enhance and supplement the properties provided by the GATA6⁺ macrophages when the pericardial space is breached and the serous fluid is expelled.

### CROSS-REFERENCES TO RELATED APPLICATIONS

This application is a continuation-in-part of U.S. application Ser. No. 16/531,263, filed on Aug. 5, 2019, which is a continuation of U.S. application Ser. No. 15/877,586, now U.S. Pat. No. 10,383,977, filed on Jan. 23, 2018, which is a division of U.S. application Ser. No. 15/386,640, now U.S. Pat. No. 10,143,778, filed on Dec. 21, 2016, which is a continuation-in-part of U.S. application Ser. No. 13/328,287, now U.S. Pat. No. 9,532,943, filed on Dec. 16, 2011, which claims the benefit of U.S. Provisional Application No. 61/425,172, filed on Dec. 20, 2010.

### FIELD OF THE INVENTION

The present invention relates to methods for treating cardiac disorders. More particularly, the present invention relates to biological formulations and methods for delivering same to treat damaged cardiac tissue and cardiac disorders.

### BACKGROUND OF THE INVENTION

As is well known in the art, the wall of the human heart comprises a continuous multi-layer tissue structure that is surrounded by a multi-membraned structure called the pericardium.

Referring to FIG. 1, on the left side of the heart 100 is the heart wall 102 of the left ventricle 104. As illustrated in FIG. 1, the heart wall 102 consists primarily of myocardium 124, which comprises a majority of the mass of the heart wall 102.

The innermost surface of the heart wall 102 comprises the endocardium 122, which lines the innermost surface of the myocardium 124. The outermost surface of the heart wall 102 comprises the pericardium 112.

As further illustrated in FIG. 1, the pericardium 112 comprises three (3) seminal layers: the visceral layer of the serous pericardium 114 (also referred to as the epicardium), the parietal layer of the serous pericardium 118 and the fibrous pericardium 120.

The pericardium 112 provides a plurality of cardio-protective properties and, hence, functions, including, (i) stabilizing the heart's position in a subject's mediastinum, (ii) lubricating the heart's movement against other physiological structures in a subject's thoracic cavity, (iii) shielding the heart from infections and (iv) preventing excessive dilation of the heart in cases of acute volume overload.

Adjacent to the pericardium 112 is the pericardial cavity or space 116, which is defined by the outer surface 126 of the visceral layer of the serous pericardium 114 and the inner surface 128 of the parietal layer of the serous pericardium 118.

Both the visceral layer of the serous and parietal layers of the serous pericardium 114, 118 produce and secrete serous fluid (also referred to as pericardial fluid) into the pericardial cavity 116. The serous fluid generally comprises a plurality of immune cells, cytokines, growth factors and miRNAs associated with pro-inflammatory and reparative responses under various physiological conditions.

As set forth in Deniset, et al., Gata6⁺ Pericardial Cavity Macrophages Relocate to the Injured Heart and Prevent Cardiac Fibrosis, Immunity, vol. 51(1), pp. 131-140 (2019), it has been found that the serous fluid further comprises a specific immune cell, i.e. a subtype of macrophages, referred to as GATA6⁺ macrophages, which provide several significant cardio-regenerative properties, including (i) facilitating the repair of damaged cardiac tissue (e.g., regeneration and remodeling of damaged cardiac tissue) and (ii) significantly reducing maladaptive remodeling characterized by fibrosis of cardiac tissue after myocardial infarction-induced cardiac injury.

As is well established, during typical open-heart surgical procedures, the pericardium is often incised and the pericardial space is breached, which can, and often will, result in the loss of the serous fluid (and, hence, GATA6⁺ macrophages) and, thus, one or more of the above noted cardio-regenerative properties associated with the pericardium.

In the United States, it is common practice for surgeons to avoid closing the pericardium and repairing the breached pericardial space after an open-heart surgical procedure to reduce the risk of pericardial effusion and, thereby, reduce the incidence of post-operative complications associated therewith, e.g., cardiac tamponade.

However, in view of emerging evidence that closing the pericardium can reduce the incidence of pericardial effusion and restore one or more of the cardio-protective properties, there is a renewed interest in closing and, thus, repairing the pericardium after an open-heart surgical procedure.

Various prostheses have thus been developed to repair the pericardium after an open-heart surgical procedure. Illustrative is the prosthetic xenograft extracellular matrix (ECM) tissue graft disclosed by Rego, et al., Pericardial Closure with Extracellular Matrix Scaffold Following Cardiac Surgery Associated with a Reduction of Postoperative Complications and 30-Day Hospital Readmissions, Journal of Cardiothoracic Surgery, vol. 14(1), p. 61 (2019).

Rego, et al. opine that the decellularized ECM tissue graft will reduce the incidence of pericardial effusion and restore some of the cardio-protective properties provided by the pericardium, i.e. shielding the heart from infections and preventing excessive dilation of the heart in cases of acute volume overload.

However, since the Rego, et al. ECM tissue graft is employed well after the pericardial space is breached and the serous fluid is expelled, the cardio-regenerative properties provided by the GATA6⁺ macrophages are lost.

There thus remains a need for improved cardiovascular prostheses to close and, thus, repair the pericardium and preserve cardio-regenerative properties provided by the GATA6⁺ macrophages in the serous fluid after an open-heart surgical procedure.

There is also a need to provide methods for delivering biological formulations to the pericardial space to (i) enhance the cardio-regenerative properties provided by the GATA6⁺ macrophages in the serous fluid or (ii) restore the cardio-regenerative properties provided by the GATA6⁺ macrophages when the pericardial space is breached and the serous fluid is expelled.

It is therefore an object of the present invention to provide improved cardiovascular prostheses that are adapted to close and, thus, repair the pericardium and preserve, enhance and/or supplement the cardio-regenerative properties provided by the GATA6⁺ macrophages in the serous fluid after an open-heart surgical procedure.

It is another object of the present invention to provide methods for delivering biological formulations to the pericardial space to (i) enhance and supplement the cardio-regenerative properties provided by the GATA6⁺ macrophages in the serous fluid or (ii) restore, enhance and supplement the cardio-regenerative properties provided by the GATA6⁺ macrophages when the pericardial space is breached and the serous fluid is expelled.

It is another object of the present invention to provide biological formulations that are adapted to enhance the properties provided by the GATA6⁺ macrophages in the serous fluid.

It is another object of the present invention to provide biological formulations that are adapted to supplement the properties provided by the GATA6⁺ macrophages in the serous fluid.

It is another object of the present invention to provide biological formulations that induce GATA6⁺ macrophage recruitment and proliferation and, thereby, enhance remodeling of damaged cardiac tissue and regeneration of new cardiac tissue and reduction of maladaptive remodeling.

It is another object of the present invention to provide biological formulations, which, when delivered proximate damaged tissue, are adapted to modulate inflammation, reduce maladaptive remodeling and induce remodeling of the damaged tissue, including neovascularization of the damaged tissue, and regeneration of new tissue and tissue structures.

It is another object of the present invention to provide biological formulations, which, when delivered to the pericardial space proximate damaged tissue, are adapted to modulate inflammation, reduce maladaptive remodeling and induce remodeling of the damaged tissue, including neovascularization of the damaged tissue, and regeneration of new tissue and tissue structures.

### SUMMARY OF THE INVENTION

The present invention is directed to biological formulations and methods for delivering same to treat damaged cardiac structures, associated tissue, and cardiac disorders.

In some embodiments of the invention, the present invention is directed to biological formulations that restore the properties provided by GATA6⁺ macrophages in serous fluid that are lost during surgical procedures, i.e. facilitating the repair of damaged cardiac tissue and reducing maladaptive remodeling.

In some embodiments of the invention, the present invention is directed to biological formulations that induce recruitment and proliferation of GATA6+ macrophages in the serous fluid and, thereby, the concentration of the GATA6+ macrophages, whereby the properties provided by the GATA6+ macrophages are enhanced.

In some embodiments of the invention, the present invention is directed to biological formulations that modulate, i.e. enhance, at least one paracrine process associated with the GATA6+ macrophages in the serous fluid.

In some embodiments of the invention, the present invention is directed to a biological formulation that supplements the properties provided by the GATA6+ macrophages in the serous fluid.

In some embodiments of the invention, the present invention is directed to methods of delivering biological formulations proximate the pericardial space of a mammalian heart that (i) enhance and supplement the properties provided by the GATA6⁺ macrophages in the serous fluid and/or (ii) restore, enhance and supplement the properties provided by the GATA6⁺ macrophages when the pericardial space is breached and the serous fluid is expelled.

Thus, in some embodiments of the invention, there is provided methods of treating damaged or diseased cardiac tissue by delivering a biological formulation to proximate the pericardial space of a mammalian heart.

In some embodiments of the invention, the biological formulations comprise a natural biological material, such as, without limitation, amniotic fluid and Wharton's jelly.

Thus, in some embodiments of the invention, there is provided a method of treating damaged cardiac tissue comprising:

(i) providing a biological formulation comprising Wharton's jelly from a mammalian source, the biological formulation being adapted to induce recruitment and proliferation of GATA6+ macrophages contained in the serous fluid when the biological formulation is delivered to a target site disposed proximate a pericardial space of a subject's heart comprising serous fluid and damaged cardiac tissue,

(ii) delivering the biological formulation to the target site in the subject's heart, wherein, after the delivery of the biological formulation to the target site, the biological formulation enhances concentration of the GATA6+ macrophages proximate the damaged tissue site, whereby the biological formulation induces modulated healing of the damaged cardiac tissue.

In some embodiments of the invention, modulated healing comprises inflammation modulation of the damaged tissue and induced neovascularization, stem cell proliferation and, thereby, positive remodeling of the damaged tissue, and regeneration of new tissue and tissue structures with site specific structural and functional properties.

In some embodiments of the invention, the biological formulations comprise ECM compositions comprising acellular ECM derived from a mammalian tissue source.

According to the invention, the mammalian tissue sources can comprise, without limitation, small intestine submucosa (SIS), urinary bladder submucosa (UBS), urinary basement membrane (UBM), liver basement membrane (LBM), stomach submucosa (SS), mesothelial tissue, placental tissue and cardiac tissue.

In some embodiments of the invention, the biological formulations include at least one additional, i.e. exogenous, biologically active agent.

In some embodiments of the invention, the biologically active agent comprises an exosome.

Thus, in some embodiments of the invention, there is also provided a method of treating damaged cardiac tissue comprising:

(i) providing an exosome augmented biological formulation comprising Wharton's jelly from a mammalian source and a plurality of exogenous exosomes, the exosome augmented biological formulation being adapted to induce recruitment and proliferation of GATA6+ macrophages contained in the serous fluid and enhance at least one paracrine process associated with the GATA6+ macrophages when the exosome augmented biological formulation is delivered to a target site disposed proximate a pericardial space of a subject's heart comprising serous fluid and damaged cardiac tissue,

(ii) delivering the exosome augmented biological formulation to the target site in the subject's heart, wherein, after the delivery of the exosome augmented biological formulation to the target site, the exosome augmented biological formulation enhances concentration of the GATA6+ macrophages proximate the damaged cardiac tissue and enhances at least one paracrine process associated with the GATA6+ macrophages, whereby the exosome augmented biological formulation induces modulated healing of the damaged cardiac tissue.

In some embodiments of the invention, the biologically active agent comprises a growth factor, such as, without limitation, basic fibroblast growth factor (bFGF), transforming growth factor-beta (TGF-β), vascular endothelial growth factor (VEGF) and hepatocyte growth factor (HGF).

In some embodiments of the invention, the biological formulations include a pharmacological agent.

In some embodiments of the invention, the pharmacological agent comprises an antibiotic agent.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration showing the various layers of a mammalian heart wall;

FIG. 2 is a schematic illustration showing the various biological processes of GATA6⁺ macrophages in a cardiac tissue microenvironment;

FIG. 3 is a schematic illustration showing the various biological processes of GATA6⁺ macrophages and a biological formulation in a cardiac tissue microenvironment, in accordance with the invention;

FIG. 4A is an illustration of a mammalian heart with a catheter system routed internally therethrough with transseptal access to the left atrium and left ventricle of the mammalian heart, in accordance with the invention;

FIG. 4B is side plan sectional view of an injector device cannula of the catheter system shown in FIG. 4A, in accordance with the invention;

FIG. 5A is an illustration of a multi-needle injector device disposed proximate a damaged cardiac tissue region, in accordance with the invention;

FIG. 5B is side plan sectional view of cannula members of the multi-needle injector device shown in FIG. 5A that are routed into and through the fibrous pericardium and parietal layer of the serous pericardium of the pericardium to access the pericardial space of a subject's heart, in accordance with the invention;

FIG. 6A is a top plan view of a sheet structure that is adapted to deliver a biological formulation proximate a pericardial space and/or damaged cardiac tissue, in accordance with the invention;

FIG. 6B is illustration of a mammalian heart showing the sheet structure shown in FIG. 6A disposed on the pericardium of the heart wall, in accordance with the invention;

FIG. 7A is a perspective view of another sheet structure that is also adapted to deliver a biological formulation proximate a pericardial space and/or damaged cardiac tissue, in accordance with the invention; and

FIG. 7B is illustration of a mammalian heart showing the sheet structure shown in FIG. 7A disposed on the pericardium of the heart wall, in accordance with the invention.

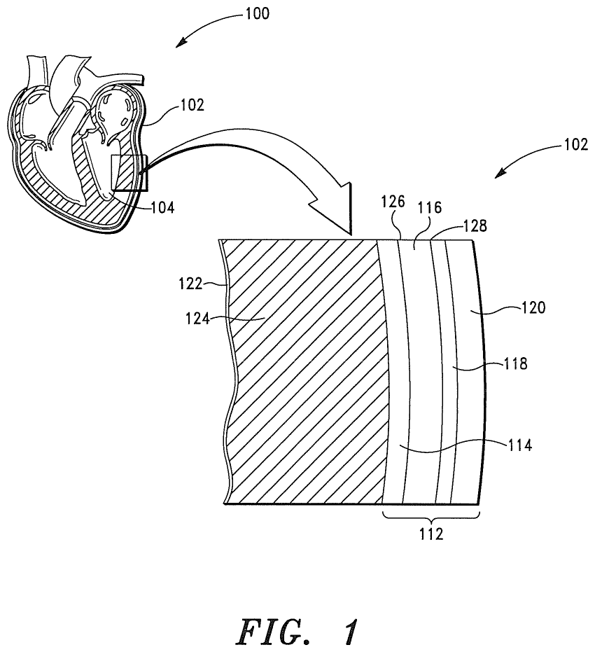

### DETAILED DESCRIPTION OF THE PREFERRED EMBODIMENT

Before describing the present invention in detail, it is to be understood that this invention is not limited to particularly exemplified compositions, structures, apparatus, and methods, as such may, of course, vary. Thus, although a number of compositions, structures, apparatus, and methods similar or equivalent to those described herein can be used in the practice of the present invention, the preferred compositions, structures, apparatus, and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference herein in their entirety.

As used in this specification and the appended claims, the singular forms “a, “an” and “the” include plural referents unless the content clearly dictates otherwise. Thus, for example, reference to “a biologically active agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

### Definitions

The terms “extracellular matrix” and “ECM” are used interchangeably herein, and mean and include a collagen-rich substance that is found in between cells in mammalian tissue, and any material processed therefrom, e.g., acellular ECM derived from mammalian tissue sources.

According to the invention, ECM can be derived from a variety of mammalian tissue sources, including, without limitation, small intestine submucosa (SIS), urinary bladder submucosa (UBS), stomach submucosa (SS), central nervous system tissue and epithelium of mesodermal origin, i.e. mesothelial tissue.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

ECM can also be derived from basement membrane of mammalian tissue/organs, including, without limitation, urinary basement membrane (UBM), liver basement membrane (LBM), and amnion, chorion, allograft pericardium, allograft dermis, amniotic membrane, Wharton's jelly, umbilical cord, and combinations thereof.

Additional sources of mammalian basement membrane include, without limitation, spleen tissue, lymph node tissue, salivary gland tissue, prostate tissue, pancreas tissue and tissue from other secreting glands.

The ECM can also be derived from dermal tissue, subcutaneous tissue, placental tissue, cardiac tissue, e.g., pericardial and/or myocardial tissue, kidney tissue, lung tissue, gastrointestinal tissue, i.e. large and small intestinal, appendix, omentum and pancreas tissue, and combinations thereof.

ECM can also be derived from other sources, including, without limitation, collagen from plant sources and synthesized extracellular matrices, i.e. cell cultures. ECM can also comprise ECM synthesized in vitro, e.g., collagen producing cell lines, and collagen and ECM from non-mammalian tissue sources, such as, without limitation, avian, reptilian, fish, and other marine sources.

The terms “decellularized” and “acellular” are used interchangeably herein in connection with ECM, and mean and include ECM derived from mammalian tissue subjected to a decellularized process and, hence, exhibits a reduced glycosaminoglycan (GAG) content and markedly altered collagen and fibronectin structures compared to naturally occurring mammalian tissue.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussusceptive angiogenesis, sprouting angiogenesis, therapeutic angiogenesis and vasculogenesis.

The term “adverse biological response”, as used herein, means and includes a physiological response that is sufficient to induce a biological process and/or restrict a phase associated with biological tissue healing in vivo, including without limitation, neovascularization and remodeling of the damaged biological tissue. The term “adverse biological response” thus includes an “adverse inflammatory response”, e.g., inflammatory responses characterized by the development of fibrotic tissue.

The term “adverse inflammatory response”, as used herein, means and includes a physiological response that is sufficient to induce clinically relevant expression of pro-inflammatory cytokines, such as interleukin-1 beta (IL-1β) and monocyte chemoattractant protein-1 (MCP-1) in vivo, and, thereby, induce a biological process and/or restrict a phase associated with biological tissue healing, including without limitation, neovascularization and remodeling of the damaged biological tissue.

The terms “maladaptive remodeling” and “negative remodeling” are used interchangeably herein, and mean and include a physiological response that is sufficient to induce a biological process associated with the remodeling of damaged biological tissue into remodeled tissue having impaired function compared to normal endogenous tissue, e.g., fibrotic tissue. Maladaptive remodeling and negative remodeling thus also include an “adverse inflammatory response”, e.g., inflammatory responses characterized by the development of fibrotic tissue.

The terms “biologically active agent” and “biologically active composition” are used interchangeably herein, and mean and include agent or composition that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors and compositions comprising same: transforming growth factor alpha (TGF-α), transforming growth factor beta (TGF-β), basic fibroblast growth factor (bFGF) (also referred to as fibroblast growth factor-2 (FGF-2)), vascular endothelial growth factor (VEGF) and hepatocyte growth factor (HGF).

The terms “biologically active agent” and “biologically active composition” also mean and include, without limitation, the following cells and compositions comprising same: myofibroblasts, mesenchymal stem cells and embryonic stem cells.

The terms “biologically active agent” and “biologically active composition” also mean and include, without limitation, the following biologically active agents (referred to interchangeably herein as a “protein”, “peptide” and “polypeptide”) and compositions comprising same: collagen (types I-V), proteoglycans, glycosaminoglycans (GAGs), glycoproteins and cytokines, such as interleukin-10 (IL-10), interleukin-la (IL-la) and interleukin-8 (IL-8).

The terms “biologically active agent” and “biologically active composition” also mean and include an “exosome”, “microsome”, “extracellular vesicle” or “micro-vesicle,” which are used interchangeably herein, and mean and include a micellar body formed from a hydrocarbon monolayer or bilayer configured to contain or encase a composition of matter, such as a biologically active agent. The terms “exosome”, “microsome”, “extracellular vesicle” and “micro-vesicle” thus include, without limitation, a micellar body formed from a lipid layer configured to contain or encase biologically active agents and/or combinations thereof.

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-fibrotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or anti-thrombotic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Additional biologically active and pharmacological agents are set forth in Co-pending priority U.S. application Ser. No. 16/531,263, which is expressly incorporated herein in its entirety.

The term “biological formulation”, as used herein, means and includes a composition comprising a non-synthetic substance, such as mammalian substance, or synthetic substance, such as a biocompatible polymer, which can also include a “phaiinacological agent” and/or a “biologically active agent” and/or any additional agent or component identified herein.

The term “therapeutically effective”, as used herein, means that the amount of the “pharmacological agent” and/or “biologically active agent” and/or “biological formulation” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “delivery” and “administration” are used interchangeably herein, and mean and include providing a “biological formulation” and/or “biologically active agent” and/or “pharmacological agent” to a treatment site, e.g., damaged biological tissue, through any method appropriate to deliver the functional composition and/or agent or combination thereof to the treatment site. Non-limiting examples of delivery methods include direct injection, percutaneous delivery and topical application at the treatment site.

The term “adolescent”, as used herein, means and includes a mammal that is preferably less than three (3) years of age.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

Although the biological formulations of the invention are described in connection with the treatment of damaged or diseased cardiac tissue, use of the biological formulations is not limited to the treatment of damaged or diseased cardiac tissue. As will readily appreciated by one having ordinary skill in the art, the biological formulations can also be employed to treat any mammalian tissue or tissue structure, including, without limitation, liver, lung, brain, esophagus, peritoneum, etc.

As set forth above, it is well established that the pericardial space of the pericardium comprises serous fluid comprising GATA6⁺ macrophages, which, as discussed above, provide several significant cardio-regenerative properties, including (i) facilitating the repair of damaged cardiac tissue and (ii) significantly reducing maladaptive remodeling by several seminal biological processes.

The noted biological processes include seminal in vivo paracrine processes that are induced and modulated by the GATA6+ macrophages in response to cardiac tissue damage, e.g., infarcted myocardium following an ischemic injury event. The in vivo paracrine processes are discussed in detail below.

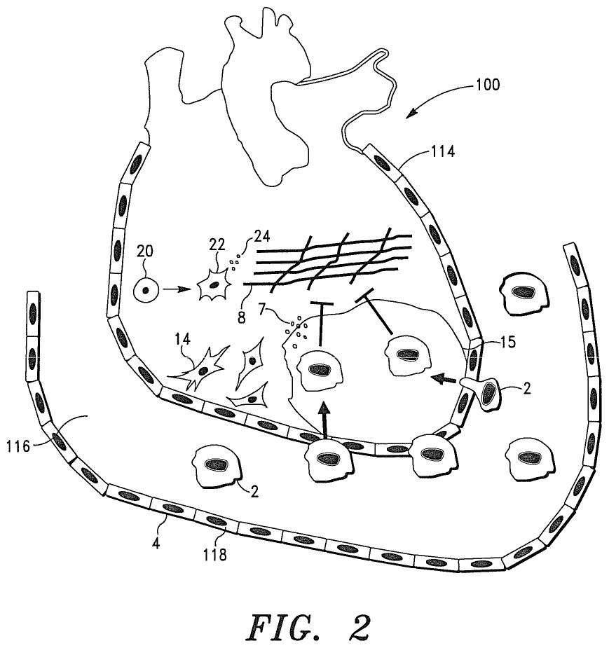

Referring to FIG. 2, when a mammalian heart 100 is subjected to an ischemic injury event that results in cardiac tissue damage, the GATA6+ macrophages 2 in the pericardial space of the heart transition from a “homeostatic” phenotype to a “cardio-regenerative” phenotype and rapidly migrate to the damaged cardiac tissue site 15.

After the GATA6+ macrophages 2 migrate to the damaged cardiac tissue site 15, the GATA6+ macrophages 2 interact with other endogenous cells recruited to the damaged cardiac tissue site to facilitate remodeling of the damaged tissue. One of the seminal cell populations that are recruited to the damaged cardiac tissue site 15 are cardiac fibroblasts 14.

As is well established, cardiac fibroblasts are one of the most abundant cell populations in a mammalian heart and are capable of synthesizing ECM components and producing cytokines that modulate homeostasis of healthy cardiac tissue. Although cardiac fibroblasts 14 are typically associated with maladaptive remodeling 8, i.e. fibrotic tissue formation, after an ischemic injury event, it is believed that when the GATA6+ macrophages interact with cardiac fibroblasts, the cardiac fibroblasts become active regulators of seminal embodiments of positive remodeling; particularly, neovascularization.

It is thus contemplated that one of the first seminal paracrine processes induced by the GATA6+ macrophages 2 is the release of a plurality of paracrine factors 7, e.g., GATA6+ macrophage-derived exosomes, TGF-β and interleukin-10 (IL-10), by the GATA6+ macrophages 6, which induce endogenous cell populations to produce increased concentrations of bioavailable growth factors, including bFGF, VEGF and hepatocyte growth factor (HGF). The cardiac fibroblasts 14 respond to the increased concentrations of bioavailable bFGF, VEGF and HGF by synthesizing ECM components and upregulating neovascularization processes that contribute to positive remodeling in vivo, and, thereby, the repair of damaged cardiac tissue.

It is also contemplated that the release of a plurality of paracrine factors 7 by GATA6+ macrophages 2 also induce the cardiac fibroblasts 14 to significantly increase expression and, thereby, synthesis of HGF in vivo. As is well established, HGF is a highly cardio-protective cytokine that inhibits apoptosis of cardiomyocytes and facilitates positive remodeling processes induced by endogenous cell populations, including the upregulation of neovascularization processes by cardiac fibroblasts.

It is also contemplated that the release of a plurality of paracrine factors 7 by GATA6+ macrophages 2 also induce local epicardial progenitor cells (EPCs) 20 to transition from an inactive state to an active state. After the EPCs 20 transition from an inactive state to an active state, the EPCs 20 undergo an epithelial-to-mesenchymal transition (EMT) and, thus, further transition from EPCs 20 having an epithelial phenotype to EPC-derived mesenchymal cells 22 (EPC-MSCs) having a mesenchymal phenotype. EPC-derived mesenchymal cells 22 contribute to positive remodeling of damaged cardiac tissue by producing and releasing additional paracrine factors 24, e.g., EPC-MSC-derived exosomes, that upregulate neovascularization processes of endogenous cells.

It is also contemplated that the release of a plurality of paracrine factors 7 by GATA6+ macrophages 2 also modulate inflammation of the damaged cardiac tissue by inducing the transition of the microenvironment of the damaged cardiac tissue site from an “acute inflammatory” state to a “wound healing” state. It is further contemplated that the transition of the microenvironment of the damaged cardiac tissue site from an “acute inflammatory” state to a “wound healing” state reduces maladaptive remodeling 8 often observed after an extended acute inflammatory immune response at the damaged cardiac tissue site.

When the microenvironment of the damaged cardiac tissue site transitions to a “wound healing” state, circulating monocyte-derived macrophages transition from a M1 subtype “acute inflammatory” macrophages to M2 subtype “wound healing” macrophages that also release paracrine factors that further modulate inflammation and contribute to positive remodeling of damaged cardiac tissue by producing and releasing additional paracrine factors, e.g., M2 macrophage-derived exosomes, that upregulate various positive remodeling processes of endogenous cells.

Thus, when a mammalian heart has a defective or damaged tissue region, e.g., infarct myocardium tissue region, the GATA6⁺ macrophages release a plurality of paracrine factors, which: (i) induce endogenous cell populations to produce increased concentrations of bioavailable growth factors, including bFGF, VEGF and HGF, (ii) induce cardiac fibroblasts to significantly increase expression and, thereby, synthesis of HGF in vivo, (iii) induce local EPCs to transition from an inactive state to an active state and undergo EMT and (iv) induce inflammation modulation of the damaged cardiac tissue by inducing the transition of the microenvironment of the damaged cardiac tissue site from an “acute inflammatory” state to a “wound healing” state, and, hence, reduce maladaptive remodeling and facilitate the repair of the damaged tissue region.

As indicated above, during typical open-heart surgical procedures, such as a coronary artery bypass procedure, the pericardium is often incised and the pericardial space breached, which can, and often will, result in the loss of the serous fluid. Thus, the GATA6+ macrophages and, hence, cardio-regenerative properties provided thereby are no longer present.

In some embodiments of the invention, the present invention is thus directed to providing biological formulations that restore properties provided by the GATA6⁺ macrophages that are lost during surgical procedures, including modulation of inflammation of damaged tissue, induced remodeling of the damaged tissue and regeneration of new tissue, and reduction of maladaptive remodeling.

In some embodiments of the invention, the present invention is directed to providing biological formulations (or compositions) that enhance the properties provided by the GATA6+ macrophages in the serous fluid.

In some embodiments of the invention, the present invention is directed to providing biological formulations (or compositions) that supplement the properties provided by the GATA6+ macrophages in the serous fluid.

In some embodiments of the invention, the present invention is directed to methods of delivering biological formulations proximate and into the pericardial space of a mammalian heart to (i) enhance and supplement the properties provided by the GATA6⁺ macrophages in the serous fluid or (ii) restore, enhance and supplement the properties provided by the GATA6⁺ macrophages when the pericardial space is breached and the serous fluid is expelled.

In some embodiments of the invention, the biological formulations comprise a natural biological material, such as, without limitation, amniotic fluid and Wharton's Jelly.

In some embodiments of the invention, the natural biological material comprises Wharton's Jelly.

In some embodiments of the invention, the biological formulations comprise ECM compositions comprising acellular ECM derived from a mammalian tissue source.

According to the invention, the mammalian tissue sources can comprise, without limitation, small intestine tissue, large intestine tissue, stomach tissue, lung tissue, liver tissue, kidney tissue, pancreas tissue, placental tissue, cardiac tissue, bladder tissue, prostate tissue, tissue surrounding growing enamel, tissue surrounding growing bone, and any fetal tissue from any mammalian organ.

In some embodiments of the invention, the mammalian tissue sources comprise urinary basement membrane (UBM), liver basement membrane (LBM), amnion, chorion, allograft pericardium, allograft dermis, amniotic membrane and umbilical cord.

In some embodiments of the invention, the mammalian tissue sources comprise, small intestine submucosa (SIS), urinary bladder submucosa (UBS), urinary basement membrane (UBM), liver basement membrane (LBM), stomach submucosa (SS), mesothelial tissue and cardiac tissue.

According to the invention, the ECM composition can comprise acellular ECM derived from one (1) mammalian tissue source or acellular ECM derived from different mammalian tissue sources.

In some embodiments of the invention, the mammalian tissue source comprises an adolescent mammalian tissue source, i.e. an adolescent mammal, such as a piglet, which is preferably less than three (3) years of age.

According to the invention, an ECM can be decellularized to provide acellular ECM by various conventional means.

According to the invention, the ECM can be decellularized via one of the conventional decellularization methods disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety.

In some embodiments of the invention, the ECM is decellularized via one of the unique Novasterilis™ processes disclosed in U.S. Pat. No. 7,108,832 and U.S. patent application Ser. No. 13/480,205; which are incorporated by reference herein in their entirety.

According to the invention, the ECM can be formed into a single component particulate structure and fluidized as described in U.S. Pat. Nos. 5,275,826, 6,579,538, 6,933,326 and 8,980,296 (which are incorporated by reference herein in their entirety) to form an ECM composition and, hence, a biological formulation of the invention.

In some embodiments of the invention, the particulate ECM is subsequently filtered to achieve a desired particulate size. According to the invention, suitable particulate ECM can have a diameter or maximum width in the range of 0.001-2000 microns (μm).

According to the invention, the ECM compositions and, hence, biological formulations can also comprise single component particulate structures comprising different ECM from different mammalian tissue sources, e.g., ECM derived from small intestine submucosa and liver basement membrane. The mammalian tissue sources can also comprise different mammalian animals or an entirely different species of mammals.

In some embodiments of the invention, the ECM particulates comprise multi-component particulate structures comprising an ECM core and outer layer (or coating), which can comprise any of the aforementioned ECMs and/or a synthetic ECM or a different material or composition, e.g., an ECM-mimicking composition, such as described U.S. application Ser. Nos. 14/832,109 and 14/832,163, which are incorporated by reference herein in their entirety.

In some embodiments of the invention, the synthetic ECM is adapted to mimic or emulate at least one seminal property of mammalian tissue-derived, non-synthetic ECM, such as the synthetic ECM materials disclosed in Applicant's U.S. Pat. No. 8,568,761, which is incorporated by reference herein in its entirety.

In some embodiments of the invention, the synthetic ECM material comprises poly(glycerol sebacate) (PGS). As set forth in Applicant's Co-pending U.S. application Ser. No. 16/531,263, PGS provides numerous beneficial structural and biochemical actions or activities when a biological formulation of the invention employing same is delivered to or disposed proximate damaged tissue.

In some embodiments of the invention, the particulate ECM is mixed with a liquid solution to form fluidized biological formulations in various forms, including, without limitation, a gel, a liquid, a paste, an emulsion, mixed liquids, mixed emulsions, mixed gels and mixed pastes.

According to the invention, the liquid solution can comprise any suitable buffer solution, including, without limitation, water and saline.

According to the invention, the concentration of the particulate ECM in a fluidized biological fommlation of the invention can range from approximately 0.001 mg/ml to 200 mg/ml. Suitable particulate ECM concentration ranges thus include, without limitation, approximately 5 mg/ml-150 mg/ml, 10 mg/ml-125 mg/ml, 25 mg/ml-100 mg/ml, 20 mg/ml-75 mg/ml, 25 mg/ml-60 mg/ml and 30 mg/ml-50 mg/ml.

As stated above, in some embodiments of the invention, the biological formulations of the invention preferably comprise at least one additional or supplemental biologically active agent or composition, i.e. an agent that induces or modulates a physiological or biological process, or cellular activity, e.g., induces proliferation, and/or growth and/or regeneration of tissue.

Suitable supplemental biologically active agents include any of the aforementioned biologically active agents, including, without limitation, the aforementioned cells and proteins.

In some embodiments of the invention, the supplemental biologically active agent comprises an exosome, i.e. an exogenous or endogenous exosome. Thus, in some embodiments of the invention, the biological formulations comprise a plurality of exosomes. Biological formulations comprising an exosome are hereinafter referred to as exosome augmented biological formulations.

As set forth in Co-pending U.S. application Ser. No. 16/531,263, exosomes comprise a lipid bilayer structure that contains or encapsulates a biologically active agent, such as a micro RNA (miRNA), e.g., miR-132 and miR-210, growth factor, e.g., TGF-β, TGF-α, VEGF, and insulin-like growth factor (IGF-I)) cytokine, e.g., interleukin-10 (IL-10), interleukin-1α (IL-1α) and interleukin-8 (IL-8)) and transcription factor.

As also set forth in Co-pending U.S. application Ser. No. 16/531,263, exosomes significantly enhance the delivery of biologically active agents to cells through two seminal properties/capabilities. The first property comprises the capacity of exosomes to shield the encapsulated biologically active agents (via the exosome lipid bilayer) from proteolytic agents, which can, and often will, degrade unshielded (or free) bioactive molecules and render the molecules non-functional in biological tissue environments.

The second property of exosomes comprises the capacity to directly and, hence, more efficiently deliver biologically active agents to endogenous cells in the biological tissue.

As is well known in the art, endogenous cells typically do not comprise the capacity to “directly” interact with “free” biologically active agents, such as growth factors. There must be additional biological processes initiated by the endogenous cells to interact directly with biologically active agents, e.g., expression of receptor proteins for or corresponding to the biologically active agents.

Exosomes facilitate direct interaction by and between endogenous cells and exosome encapsulated biologically active agents (and, hence, direct delivery of bioactive molecules to endogenous cells), which enhances the bioactivity of the agents.

According to the invention, when an exosome augmented biological formulation comprises acellular ECM and the exosome augmented biological formulation is delivered proximate to the damaged tissue; particularly, damaged cardiac tissue, the exosome augmented biological formulation “concomitantly” induces a multitude of significant biological processes in vivo, including significantly enhanced (i) inflammation modulation of the damaged tissue, (ii) neovascularization inducement, (iii) stem cell proliferation inducement, (iv) inducement of remodeling of the damaged tissue, and (v) inducement of regeneration of new tissue and tissue structures with site-specific structural and functional properties, compared to acellular ECM alone.

By way of example, when an exosome augmented biological formulation comprising encapsulated miR-210 is disposed proximate damaged tissue, the exosome augmented biological formulation facilitates the release and production of endogenous angiogenic cytokines (e.g., IL-1α and tumor necrosis factor-α (TNF-α)), downregulates apoptotic genes (e.g., protein tyrosine phosphatase-1B (Ptp1b)), which, thereby, significantly enhances neovascularization, including angiogenesis, stem cell proliferation, suppression of host cell apoptosis, remodeling of the damaged tissue, and regeneration of new tissue and tissue structures.

The enhanced stem cell proliferation is induced via the delivery of exosome encapsulated miRNAs and transcription factors to the damaged tissue, which signals the endogenous stem cells to bind and/or attach to the acellular ECM and proliferate.

By way of further example, when an exosome augmented biological formulation comprising encapsulated IL-8 is disposed proximate damaged tissue, the exosome encapsulated IL-8 and, hence, exosome augmented biological formulation modulates the transition of M1 type “acute inflammatory” macrophages to M2 type “wound healing” macrophages initiated by the acellular ECM.

In some embodiments of the invention, the exosomes are derived and, hence, processed from an aforementioned tissue source. In some embodiments, the exosomes are processed and derived from a mammalian fluid composition including, but not limited to blood, amniotic fluid, lymphatic fluid, interstitial fluid, pleural fluid, peritoneal fluid, pericardial fluid and cerebrospinal fluid.

In some embodiments of the invention, exosomes are derived and, hence, processed from in vitro or in vivo cultured cells.

According to the invention, exosomes can be derived (or isolated) from any cell source, including any one of the aforementioned cells.

The exosomes can also be derived from one of the following cell sources: cardiac progenitor cells (CPCs), valvular interstitial cells (VICs), amniotic fluid-derived (e.g., amniotic fluid-derived mesenchymal stem cells (af-MSCs) and embryonic-like stem cells), placental cells, umbilical cord-derived mesenchymal stem cells (uc-MSCs), Wharton's jelly-derived mesenchymal stem cells (wj-MSCs), amniotic membrane-derived mesenchymal stem cells (am-MSCs), adipose tissue-derived mesenchymal stem cells (at-MSCs) and bone marrow-derived mesenchymal stem cells (bm-MSCs).

According to the invention, the exosomes can be isolated from any of the aforementioned cell sources, tissue sources, mammalian fluid compositions and combinations thereof using any conventional processing method, such as the processing method disclosed in Andriolo, et al., Exosomes from Human Cardiac Progenitor Cells for Therapeutic Applications: Development of a GMP-Grade Manufacturing Method, Frontiers in Physiology, vol. 9, p. 1169 (2018), which is incorporated by reference herein in its entirety.

According to the invention, any of the aforementioned cells can be cultured in a cell culture media under hypoxic conditions to induce a higher production rate of exosomes.

In some embodiments of the invention, at least two or more of the aforementioned cell sources are co-cultured under hypoxic conditions to induce production of a population of exosomes comprising a plurality of various subtypes, e.g., am-MSCs co-cultured with cardiac myocytes under hypoxic conditions to produce a distributed population of am-MSC exosomes and cardiac myocytes exosomes.

The aforementioned cells can also be cultured on one of the aforementioned ECMs. According to the invention, the cells condition the ECM by releasing exosomes that bind to the ECM.

In some embodiments of the invention, the exosomes comprise semi-synthetically generated exosomes. According to the invention, the semi-synthetically generated exosomes can be derived from an exosome producing cell line, including one or more of the aforementioned cell sources.

By way of example, semi-synthetically generated exosomes can be generated by incubating mesenchymal stem cells in a medium comprising a predetermined concentration of any one of the aforementioned biologically active agents and/or pharmacological agents and, after a predetermined period of time, removing the mesenchymal stem cells from the incubating medium and in vitro culturing the cells using conventional cell culture techniques. The cell culture media employed can then be processed to isolate one or more exosome-encapsulated biologically active agents and/or pharmacological agents.

According to the invention, the exosome-encapsulated biologically active agents and/or pharmacological agents can be isolated from the cell culture media using any known conventional method, such as ultra-centrifugation.

According to the invention, the semi-synthetically generated exosomes markedly improve the efficacy of the aforementioned biologically active agents and/or the pharmacological agents by providing a means of traversing the cell membrane of endogenous cells.

In one embodiment of the invention, when an exosome augmented biological formulation of the invention comprises Wharton's jelly and an exosome derived from a mesenchymal stem cell, such as an am-MSC, at-MSC and uc-MSC, a CPC or VIC, and the exosome augmented biological formulation is delivered to the pericardial space of a mammalian heart and, hence, serous fluid contained therein, the exosome augmented biological formulation enhances the cardio-regenerative properties provided by the GATA6⁺ macrophages; particularly, reducing maladaptive remodeling and inducing and/or supporting repair of damaged tissue.

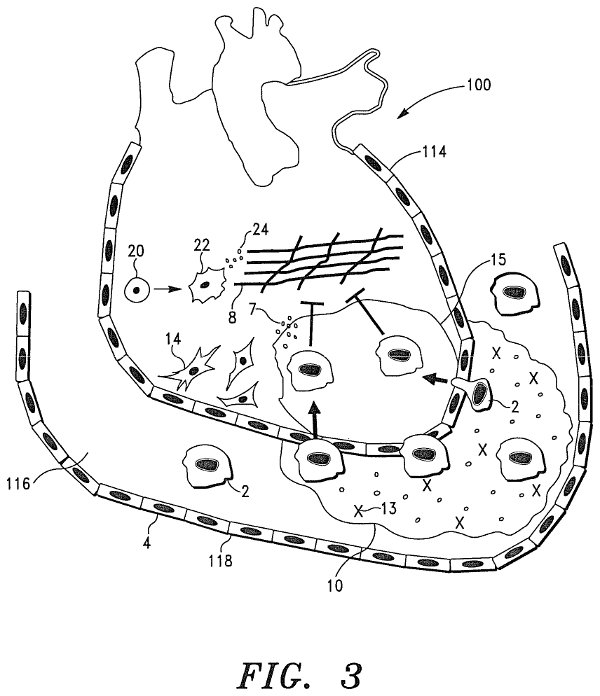

Referring now to FIG. 3, according to the invention, in one embodiment of the invention, when an exosome augmented biological formulation 10 is delivered to the pericardial space 116 of a mammalian heart 100 that is proximate a damaged cardiac tissue site 15, the exosome augmented biological formulation 10 enhances the paracrine processes (and cardio-regenerative properties associated therewith) induced by the GATA6⁺ macrophages 2 that migrate to the damaged cardiac tissue site 15, including (i) inducing endogenous cell populations to produce increased concentrations of bioavailable growth factors, including bFGF, VEGF and HGF, (ii) inducing cardiac fibroblasts to significantly increase expression and, thereby, synthesis of HGF in vivo, (iii) inducing local EPCs to transition from an inactive state to an active state and undergo EMT and (iv) inducing transition of the microenvironment of the damaged cardiac tissue site from an “acute inflammatory” state to a “wound healing” state.

According to the invention, the exosome augmented biological formulation 10 enhances the cardio-regenerative properties provided by the GATA6⁺ macrophages via several significant induced biological processes discussed below.

One of the seminal biological processes provided by the exosome augmented biological formulation 10 comprises the release of growth factors 13, i.e. bFGF, VEGF and HGF (among other proteins and cytokines) from the Wharton's jelly component of the exosome augmented biological formulation 10. The release of growth factors 13 from the Wharton's jelly component further increases the concentrations of bioavailable growth factors in vivo and, thus, similarly induces endogenous cells to produce increased concentrations of bioavailable growth factors, including bFGF, VEGF and HGF.

As discussed above, the endogenous cardiac fibroblasts 14 respond to the increased concentrations of bioavailable bFGF, VEGF and HGF by synthesizing ECM components and upregulating neovascularization processes that contribute to positive remodeling in vivo, and, thereby, the repair of damaged cardiac tissue.

Further seminal biological processes provided by the exosome augmented biological formulation 10 result from the interaction by and between the GATA6+ macrophages 2 and the exosome augmented biological formulation 10.

It is contemplated that, by virtue of the high hyaluronic acid content of the Wharton's jelly component of the exosome augmented biological formulation 10, the Wharton's jelly modulates the tissue microenvironment in response to cardiac tissue damage and, thereby, directly and/or indirectly enhances the aforementioned seminal paracrine processes of the GATA6⁺ macrophages 2. Indeed, Wharton's jelly also comprises native exosomes and mesenchymal stem cells that will also directly and/or indirectly enhance the aforementioned seminal paracrine processes of the GATA6⁺ macrophages 2.

In some embodiments of the invention, the additional exosomes 13 of the exosome augmented biological formulation 10 will also directly and/or indirectly enhance the aforementioned seminal paracrine processes of the GATA6⁺ macrophages 2.

It is further contemplated that the exosome augmented biological formulation 10 (i.e. the Wharton's jelly component and additional exosome component 13 of the formulation) in combination with the paracrine factors 7 released by the GATA6⁺ macrophages 2 induces the cardiac fibroblasts 14 to further increase expression and, thereby, synthesis of HGF in vivo.

As discussed above, HGF is a highly cardio-protective cytokine that inhibits apoptosis of cardiomyocytes and facilitates positive remodeling processes induced by endogenous cell populations, including the upregulation of neovascularization processes by cardiac fibroblasts.

It is further contemplated that recruitment and activation of epicardial progenitor cells (EPCs) is enhanced. As discussed above, when the EPCs 20 transition from an inactive to an active state, the EPCs 20 undergo an epithelial-to-mesenchymal transition (EMT) and, thus, further transition from EPCs 20 having an epithelial phenotype to EPC-derived mesenchymal cells 22 (EPC-MSCs) having a mesenchymal phenotype. EPC-derived mesenchymal cells 22 contribute to positive remodeling of damaged cardiac tissue by producing and releasing additional paracrine factors 24, e.g., EPC-MSC-derived exosomes, that upregulate neovascularization processes of endogenous cells.

It is also contemplated that multiple inflammation modulating factors, e.g., paracrine factors 7 are released by the GATA6⁺ macrophages 6, Wharton's jelly, and additional exosomes, to induce the transition of the microenvironment of the damaged cardiac tissue site from an “acute inflammatory” state to a “wound healing” state.

It is also contemplated that the exosome augmented biological formulation 10 provides a plurality of additional paracrine processes that complement and, in some instances, enhance the paracrine processes provided by the GATA6⁺ macrophages 2 (and cardio-regenerative properties associated therewith).

It is further believed that one of the additional paracrine processes that are provided by the exosome augmented biological formulation 10 includes the inhibition of apoptosis of endogenous cells, e.g., cardiac fibroblasts 14 and cardiomyocytes, when the endogenous cells are exposed to one or more biological stressors, such as an ischemic injury event. By inhibiting the apoptosis of cardiac fibroblasts, it is further contemplated that the exosome augmented biological formulation 10 further enhances the paracrine processes of the GATA6⁺ macrophages 2 by inhibiting the apoptosis of cardiac fibroblasts 14, which allows greater populations of cardiac fibroblasts 14 to increase expression and, thereby, synthesis of HGF in vivo.

In some embodiments of the invention, when an exosome augmented biological formulation of the invention comprises Wharton's jelly and an exosome derived from a mesenchymal stem cell, such as an am-MSC, at-MSC and uc-MSC, a CPC or VIC, and the exosome augmented biological formulation is delivered to the pericardial space of a mammalian heart, where the serous fluid has been lost via a surgical procedure, e.g., an open-heart surgical procedure, the exosome augmented biological formulation at least restores the cardio-regenerative properties that were originally provided by the GATA6⁺ macrophages, i.e. reduced maladaptive remodeling and induced and/or supported repair of damaged tissue.

In some embodiments, the supplemental biologically active agent comprises a growth factor, such as, without limitation, a transforming growth factor-alpha (TGF-α), transforming growth factor-beta (TGF-β), basic fibroblast growth factor (bFGF), vascular endothelial growth factor (VEGF) and hepatocyte growth factor (HGF).

In some embodiments of the invention, the wt. % of the biologically active agent in the biological formulations of the invention, is sufficient to induce or modulate a physiological or biological process in a subject when delivered thereto, without inducing an adverse inflammatory response characterized by clinically relevant expression of pro-inflammatory cytokines, such as interleukin-1 beta (IL-1β) and monocyte chemoattractant protein-1 (MCP-1).

In a preferred embodiment of the invention, when a biological formulation is delivered to or disposed proximate damaged or diseased tissue, “modulated healing” is effectuated.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic, i.e. wound healing, inflammation, and their interplay with each other.

In such an instance, a minor amount of inflammation may ensue in response to tissue injury, but this level of inflammation response, e.g., platelet and/or fibrin deposition, is substantially reduced when compared to inflammation that takes place in the absence of a biological formulation of the invention.

By way of example, in some embodiments of the invention, a biological formulation of the invention is specifically formulated (or designed) to alter, delay, retard, reduce, and/or detain one or more of the phases associated with healing of damaged tissue in vivo, including, but not limited to, the inflammatory phase (e.g., platelet or fibrin deposition), proliferative phase and maturation phase.

In some embodiments of the invention, “modulated healing” refers to the ability of a biological formulation of the invention to alter a substantial inflammatory phase (e.g., platelet or fibrin deposition) at the beginning of the tissue healing process.

As used herein, the phrases “alter a substantial inflammatory phase”, “modulate inflammation” and “inflammation modulation” refer to the ability of a biological formulation of the invention to substantially reduce an adverse inflammatory response at an injury site and induce “wound healing”, immune responses.

In some embodiments of the invention, the term “modulated healing” also refers to the ability of a biological formulation of the invention to modulate inflammation of damaged biological tissue in vivo by reducing the infiltration of “acute inflammatory” M1 macrophages and increasing the migration and, hence, population of “wound healing” M2 macrophages.

In some embodiments of the invention, the term “modulated healing” refers to the ability of a biological formulation of the invention to induce GATA6⁺ macrophage recruitment and proliferation.

In some embodiments of the invention, the term “modulated healing” refers to the ability of a biological formulation of the invention to enhance the properties provided by GATA6+ macrophages in serous fluid.

In some embodiments of the invention, the term “modulated healing” refers to the ability of a biological formulation of the invention to (i) induce endogenous cell populations to produce increased concentrations of bioavailable growth factors, including bFGF, VEGF and HGF, (ii) induce cardiac fibroblasts to significantly increase expression and, thereby, synthesis of HGF in vivo, and (iii) induce local EPCs to transition from an inactive state to an active state and undergo EMT.

In some embodiments of the invention, “modulated healing” refers to the ability of a biological formulation of the invention to induce neovascularization, including vasculogenesis, angiogenesis, and intussusception, host cell and/or tissue proliferation, remodeling of damaged biological tissue, and regeneration of new tissue and tissue structures with site-specific structural and functional properties in vivo.

In some embodiments of the invention, the biological formulation comprises at least one pharmacological agent or composition (or drug), i.e. an agent or composition that is capable of producing a desired biological effect in vivo, e.g., stimulation or suppression of apoptosis, stimulation or suppression of an immune response, etc.

According to the invention, suitable pharmacological agents and compositions include any of the aforementioned agents, including, without limitation, antibiotics, anti-fibrotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or anti-thrombotic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

In a preferred embodiment of the invention, the HMG-CoA reductase inhibitor comprises cerivastatin, i.e. (3R,5S,6E)-7-[4-(4-fluorophenyl)-5-(methoxymethyl)-2,6-bis(propan-2-yl)py-ridin-3-yl]-3,5-dihydroxyhept-6-enoic acid.

According to the invention, when a biological formulation of the invention comprises acellular ECM and a statin; particularly, cerivastatin, and the biological formulation is delivered to or disposed proximate damaged tissue, the biological formulation induces several beneficial biochemical actions or activities, which enhance modulated healing.

The beneficial biochemical actions or activities induced when a statin augmented biological formulation is disposed proximate damaged tissue; particularly, damaged cardiac tissue, are disclosed in Applicant's U.S. Pat. No. 9,119,899 and Co-pending application Ser. No. 16/531,263.

A significant biochemical action that is induced when a statin augmented ECM composition of the invention is disposed proximate damaged biological tissue is restricted expression of MCP-1 and C-C chemokine receptor type 2 (CCR2), which provides an enhanced level of inflammation modulation of the damaged biological tissue.

Thus, in some embodiments of the invention, the term “modulated healing” also refers to the ability of a biological formulation of the invention to modulate inflammation of damaged tissue in vivo by, among other actions, restricting expression of MCP-1 and CCR2.

In some embodiments of the invention, the pharmacological agent comprises an antibiotic or antibiotic agent.

According to the invention, suitable antibiotics include any of the aforementioned antibiotics.

In some embodiments of the invention, the biological formulations preferably comprise a plurality of antibiotics.

In a preferred embodiment of the invention, the biological formulations comprise vancomycin and gentamicin.

In some embodiments of the invention, when a biological formulation of the invention comprises an antibiotic, i.e. an antibiotic augmented biological formulation, and the antibiotic augmented biological formulation is delivered to or disposed proximate damaged tissue, the antibiotic augmented biological formulation enhances inflammation modulation of the damaged tissue and, thereby, significantly enhances modulated healing of the damaged tissue.

In some embodiments of the invention, when a biological formulation of the invention comprises a plurality of antibiotics; specifically vancomycin and gentamicin, and the antibiotic augmented biological formulation is delivered to or disposed proximate damaged tissue, the antibiotic augmented biological formulation induces anti-microbial and anti-biofilm activity, which also significantly enhance inflammation modulation of the damaged tissue and, thereby, modulated healing of the damaged tissue.

Thus, in some embodiments of the invention, “modulated healing” also refers to the ability of a biological formulation of the invention to induce anti-microbial and anti-biofilm activity and, thereby, enhanced inflammation modulation of damaged tissue.

According to the invention, the biological formulations of the invention can be delivered to a mammalian heart to treat various cardiac disorders, including, without limitation, damaged or diseased biological tissue, e.g., infarct tissue, atrial fibrillation (pre- and post-operative) and the root causes thereof, and damaged and diseased mammalian organs and structures, including, without limitation, cardiac vessels and valves, such as bicuspid, tricuspid and pulmonary valves, myocardium, pericardium, arteries, veins, trachea, esophagus, etc.

In some embodiments of the invention, the biological formulations are delivered to or disposed proximate damaged or diseased biological tissue, e.g., infarct tissue, to induce modulated healing of the damaged tissue.

In some embodiments of the invention, the biological formulations are delivered to the pericardial space of the heart to treat a cardiac disorder.

In some embodiments of the invention, the biological formulations are delivered to the pericardial space proximate damaged or diseased biological tissue, e.g., infarct tissue, to induce modulated healing of the damaged tissue.

According to the invention, the biological formulations of the invention can be delivered to the pericardial space of the heart and/or disposed proximate damaged tissue of a mammalian heart via various delivery means, i.e. apparatus, systems and prostheses, and associated methods.

As discussed in detail below, suitable delivery means include, without limitation, a catheter system, direct injection with a single or multi-needle device, and prosthetic sheet structures, such the graft prostheses disclosed in Applicant's U.S. Pat. Nos. 8,877,224, 8,778,012, 9,149,496, and 10,143,778, and U.S. application. Ser. Nos. 16/531,263, 16/418,063, 14/566,359, 14/953,548 and 14/566,306, which are incorporated by reference herein.

Several exemplar delivery means, i.e. apparatus, systems and prostheses, and associated methods for delivering a biological formulation of the invention to a pericardial space of the heart and/or disposed proximate damaged tissue of a mammalian heart will now be described in detail.

### Catheter Systems

According to the invention, the biological formulations of the invention can be delivered to the pericardial space of the heart and/or disposed proximate damaged tissue of a mammalian heart via a catheter system that is configured to provide transatrial and/or transseptal access to the heart.

In some embodiments of the invention, the biological formulations are delivered to the pericardial space (and/or disposed proximate damaged tissue of a mammalian heart) using a minimally invasive, percutaneous catheter system and associated method, such as the transatrial access method disclosed in Verrier, et al., Transatrial Access to the Normal Pericardial Space: A Novel Approach for Diagnostic Sampling, Pericardiocentesis, and Therapeutic Interventions, Circulation, vol. 98(21), pp. 2331-2333 (1998), which is incorporated by reference herein in its entirety.

In some embodiments of the invention, the biological formulations are delivered to the pericardial space (and/or disposed proximate damaged tissue of a mammalian heart) using a minimally invasive, percutaneous transseptal access catheter system and associated method. According to the invention, suitable transseptal access catheter systems include, without limitation, the transseptal access catheter systems disclosed in Applicant's Co-pending U.S. application. Ser. Nos. 16/193,669, 16/238,730 and 16/553,570, which are incorporated by reference herein in their entirety.

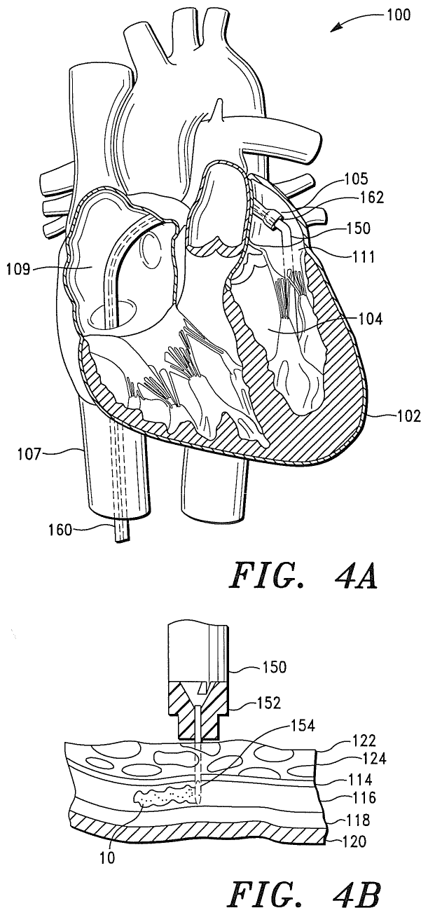

Referring now to FIG. 4A, a further suitable percutaneous transseptal access catheter system 160 and an associated method for delivering a biological formulation of the invention to a pericardial space of the heart and/or disposed proximate damaged tissue of a mammalian heart will now be described in detail.

As illustrated in FIG. 4A, the catheter system 160 comprises a formulation transfer line 150 of an injector device (not shown), which is guided into and through the lumen 162 of the transfer line 150.

As further illustrated in FIG. 4A, the formulation transfer line 150 is routed up the inferior vena cava 107 to the right atrium 109, into and through a predetermined region of the atrial septum (not shown) and into the left atrium 105 of the heart 100.

Referring now to FIG. 4B, there is shown the distal end 152 of the formulation transfer line 150. As illustrated in FIG. 4B, the distal end 152 of the formulation transfer line 150 comprises a cannula 154 that is in operative communication with the distal end 152 of the formulation transfer line 150.

To facilitate delivery of a biological formulation of the invention to the pericardial space 116 of the heart 100 (or proximate a damaged tissue region thereof), the formulation transfer line 150 is preferably disposed proximate the heart wall 102 in a manner that allows the cannula 154 to be guided into and through the endocardium 122, myocardium 124 and the epicardium 114 of the heart wall 102.

### Direct Injection Systems

A further method that can be employed to deliver a biological formulation of the invention to the pericardial space of the heart and/or disposed proximate damaged tissue of a mammalian heart comprises direct injection with a single or multi-needle device.

According to the invention, various conventional injection apparatus and systems that facilitate direct injection of a formulation or composition into and through biological tissue can be employed to deliver a biological formulation of the invention to the pericardial space of the heart and/or disposed proximate damaged tissue of a mammalian heart.

In some embodiments of the invention, a biological formulation of the invention is delivered to the pericardial space 116 of the heart 100 (or proximate a damaged tissue region thereof) via a single needle injector apparatus.

According to the invention, suitable single needle injector apparatus and systems include, without limitation, the single needle injector apparatus and system disclosed in U.S. Pat. No. 6,106,500, which is incorporated by reference herein.

In some embodiments of the invention, a biological formulation of the invention is delivered to the pericardial space 116 of the heart 100 (or proximate a damaged tissue region thereof) via a multi-needle injector device.

Suitable multi-needle injector devices are disclosed in Applicant's U.S. application Ser. No. 14/031,630, which is incorporated by reference herein in its entirety.

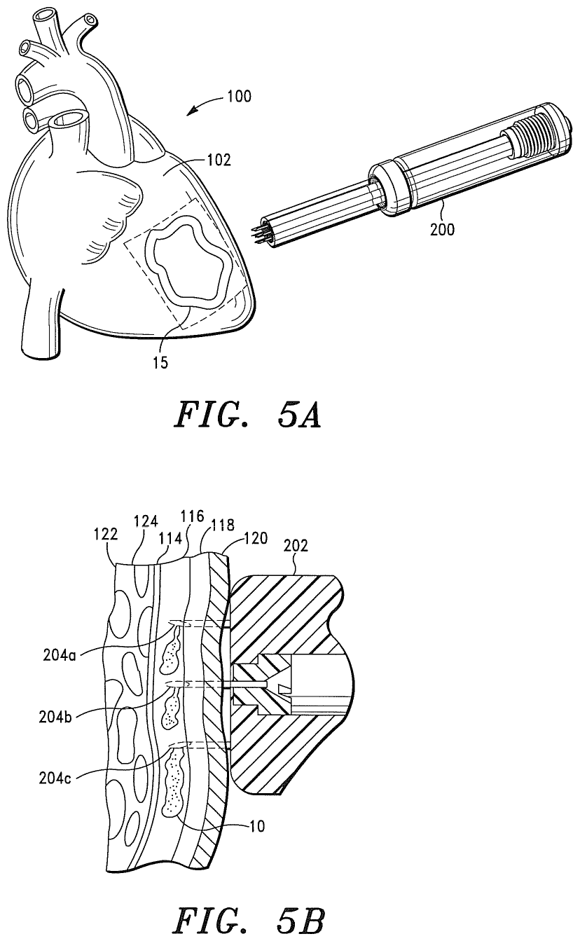

Referring now to FIG. 5A, there is shown a suitable multi-needle injector apparatus 200 that is configured to deliver a biological formulation of the invention 10 to the pericardial space 116 of the heart 100 and/or to a target tissue site proximate a damaged tissue region 15 of the heart wall 102.

Referring to FIG. 5B, the distal end 202 of the multi-needle injector apparatus 200 comprises a plurality of cannula members 204a, 204b, 204c that are in operative communication with the distal end 202 of the apparatus 200.

As illustrated in FIG. 5B, to deliver the biological formulation 10 to the pericardial space 116 of the heart 100, the multi-needle injector apparatus 200 is disposed proximate the heart wall 102 in a manner that allows the cannula members 204a, 204b, 204c of the injector apparatus 200 to be guided into and through the fibrous pericardium 120 and parietal layer of the serous pericardium 118 to access the pericardial space 116 of the heart 100.

According to the invention, the multi-needle injector apparatus 200 can also be disposed or positioned proximate a damaged tissue region of the heart 100, infarct myocardium region, in a manner that allows the cannula members 204a, 204b, 204c of the multi-needle injector apparatus 200 to deliver the biological formulation 10 directly thereto.

### Sheet Structures

According to the invention, the biological formulations of the invention can also be delivered to the pericardial space of the heart and/or disposed proximate damaged tissue of a mammalian heart via a sheet structure comprising a biological formulation of the invention. In some embodiments of the invention, the sheet structures are also configured to repair a damaged pericardium.

According to the invention, various sheet structures, such as the graft prostheses disclosed in Applicant's U.S. Pat. Nos. 8,877,224, 8,778,012, 9,149,496, and 10,143,778, and U.S. application Ser. Nos. 16/531,263, 16/418,063, 14/566,359, 14/953,548 and 14/566,306, can be employed to deliver a biological formulation of the invention to the pericardial space of the heart and/or disposed proximate damaged tissue of a mammalian heart.

Further suitable graft prostheses are disclosed in Applicant's U.S. Pat. Nos. 9,700,654, 9,694,105, 9,694,104 and 9,744,261, which are incorporated by reference herein.

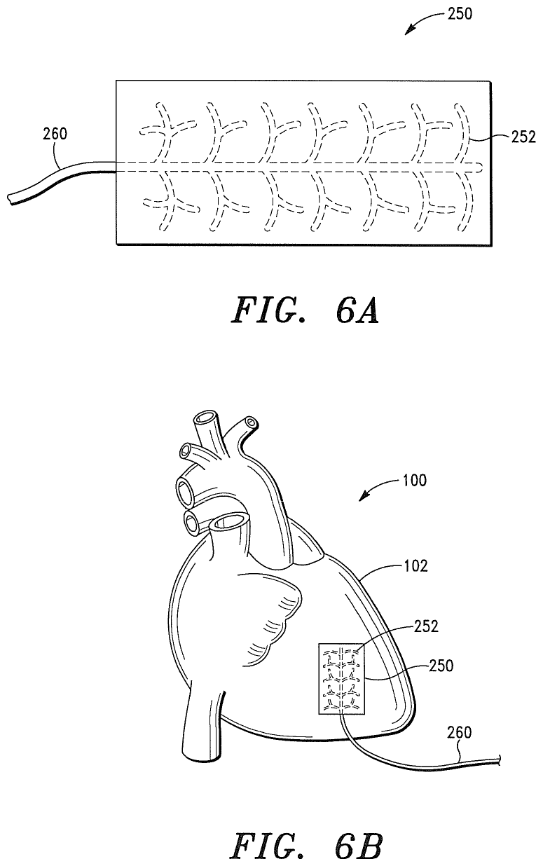

Referring now to FIGS. 6A and 6B, there is shown one embodiment of a graft prosthesis disclosed in Applicant's U.S. Pat. No. 9,700,654. As illustrated in FIG. 6B, the graft structure 250 comprises an agent dispersal network 252, which is configured to receive a biological formulation therein. The graft structure 250 further comprises a formulation input line 260 that is in communication with the agent dispersal network 252 and configured to transfer the biological formulation thereto.

As set forth in detail in U.S. Pat. No. 9,700,654, the agent dispersal network 252 includes an external delivery line (extending out of on side of the graft structure 250) that is in communication the agent dispersal network 252 and configured to deliver the biological formulation to a desired target region, i.e. directly into the pericardial space, proximate the pericardial space and/or proximate a damaged tissue region.

As also set forth in U.S. Pat. No. 9,700,654, the agent dispersal network 252 can also comprise at least one layer of a flexible, permeable liner or coating material that is configured to maintain the structural integrity of the agent dispersal network 252.

To facilitate delivery of the biological formulation directly into the pericardial space, proximate the pericardial space or proximate a damaged tissue region, the graft structure 250 is simply disposed proximate the pericardial space or the damaged tissue region, such as shown in FIG. 6A.

According to the invention, the graft structure can also be disposed over an incision site formed in the pericardium of the heart 100 to close and, thus, repair the pericardium after an open-heart surgical procedure.

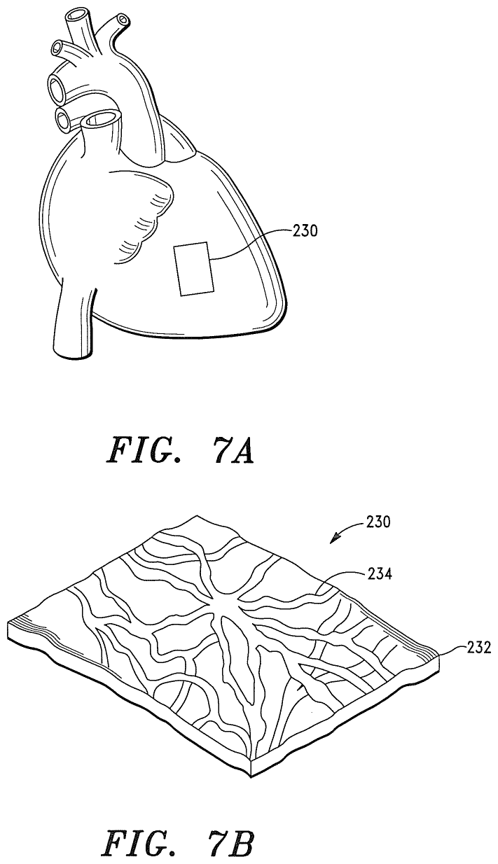

Referring now to FIGS. 7A and 7B, there is shown one embodiment of a graft prosthesis disclosed in Applicant's U.S. Pat. Nos. 9,694,105, 9,694,104 and 9,744,261.

As illustrated in FIG. 7B, the graft prosthesis 230 comprises a base graft member 232, and an internal vasculature 234, which is configured to receive a biological formulation therein.

According to the invention, the biological formulation can be infused into the internal vasculature 234 and/or incorporated in the base graft member 232.

To facilitate delivery of the biological formulation directly into the pericardial space, proximate the pericardial space or proximate a damaged tissue region, the graft prosthesis 230 is simply disposed proximate the pericardial space or the damaged tissue region, such as shown in FIG. 7A.

According to the invention, the graft prosthesis can similarly be disposed over an incision site formed in the pericardium of the heart 100 to close and, thus, repair the pericardium after an open-heart surgical procedure.

As will readily be appreciated by one having ordinary skill in the art, the present invention provides several significant unique formulations, methods and associated means for treating damaged cardiac tissue, including, without limitation, the provision of the following:

(i) improved cardiovascular prostheses that are adapted to close and, thus, repair the pericardium and preserve, enhance and/or supplement the cardio-regenerative properties provided by the GATA6⁺ macrophages in the serous fluid after an open-heart surgical procedure;

(ii) methods for delivering biological formulations to the pericardial space that (i) enhance and supplement the cardio-regenerative properties provided by the GATA6⁺ macrophages in the serous fluid and/or (ii) restore, enhance and supplement the cardio-regenerative properties provided by the GATA6⁺ macrophages when the pericardial space is breached and the serous fluid is expelled;

(iii) biological formulations that are adapted to enhance the properties provided by the GATA6⁺ macrophages in the serous fluid;

(iv) biological formulations that are adapted to supplement the properties provided by the GATA6⁺ macrophages in the serous fluid;

(v) biological formulations that induce GATA6⁺ macrophage recruitment and proliferation and, thereby, enhance remodeling of damaged cardiac tissue and regeneration of new cardiac tissue and reduction of maladaptive remodeling;

(vi) biological formulations, which, when delivered proximate damaged tissue, are adapted to modulate inflammation, reduce maladaptive remodeling and induce remodeling of the damaged tissue, including neovascularization of the damaged tissue, and regeneration of new tissue and tissue structures; and

(vii) biological formulations, which, when delivered to the pericardial space proximate damaged tissue, are adapted to modulate inflammation, reduce maladaptive remodeling and induce remodeling of the damaged tissue, including neovascularization of the damaged tissue, and regeneration of new tissue and tissue structures.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for treating damaged cardiac tissue, comprising:
providing a biological formulation comprising acellular extracellular matrix (ECM) from a mammalian source,
said biological formulation being adapted to induce recruitment and proliferation of endogenous GATA6+ macrophages, when said biological formulation is delivered to a target site disposed in a pericardial space of a subject's heart, said pericardial space comprising serous fluid,
delivering said biological formulation to said target site in said subject's heart, said target site also being disposed proximate a damaged tissue region,
wherein, after said delivery of said biological formulation to said target site, said biological formulation induces recruitment and proliferation of first endogenous GATA6+ macrophages disposed proximate said damaged tissue site, whereby said biological formulation induces modulated healing of damaged tissue in said damaged tissue region.

2. The method of claim 1, wherein said pericardial space is disposed between an outer surface of a visceral layer of a serous pericardium and an inner surface of a parietal layer of said serous pericardium.

3. The method of claim 1, wherein said mammalian tissue source is selected from the group consisting of small intestine submucosa, urinary bladder submucosa, urinary basement membrane, liver basement membrane, stomach submucosa, mesothelial tissue, placental tissue and cardiac tissue.

4. The method of claim 1, wherein said modulated healing comprises inflammation modulation of said damaged tissue and induced neovascularization of said damaged tissue, stem cell proliferation and, thereby, positive remodeling of said damaged tissue, and regeneration of new tissue and tissue structures with site specific structural and functional properties.

5. The method of claim 1, wherein said biological formulation comprises an exogenous growth factor selected from the group consisting of basic fibroblast growth factor (bFGF), transforming growth factor-beta (TGF-β), vascular endothelial growth factor (VEGF) and hepatocyte growth factor (HGF).

6. The method of claim 1, wherein said biological formulation comprises a plurality of exogenous exosomes.

7. The method of claim 1, wherein said biological formulation comprises an antibiotic agent selected from the group consisting of vancomycin and gentamicin.

8. The method of claim 1, wherein said biological formulation comprises a cytokine.

9. The method of claim 8, wherein said cytokine comprises an interleukin selected from the group consisting of interleukin-10 (IL-10), interleukin-1α (IL-1α) and interleukin-8 (IL-8)
